(19) United States
(12) Patent Application Publication
(10) Pub. No.: US 2001/0031933 A1
(43) Pub. Date: Oct. 18, 2001

(54) Disposable bladder

(76) Inventor: Gerald Asuncion Cannon, Annandale, VA (US)

Correspondence Address: Gerald Cannon, 8707 Chapel Drive, Annandale, VA, 22003, US

(21) Appl. No.: 09/734,516

(22) Filed: Dec. 12, 2000

Related U.S. Application Data
(60) Provisional application No. 60/176,037, filed on Jan. 14, 2000.

(51) Int. Cl. A61F 5/44

(52) U.S. Cl. 600/580; 604/349

(57) ABSTRACT

This is a disposable, male incontinence device, which is inexpensive and simplifies prior art. Different reservoir sizes account for minor, mild, or total incontinence, thus allowing the wearer to tailor fit the appropriate size to his incontinence level. The elastic, permeable tube rolls out over the entire penis allowing for a cool, dry, snug, custom fit.

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### REFERENCE TO A MICROFICHE APPENDIX

“Not Applicable”

### BACKGROUND OF THE INVENTION

This invention seeks to address the needs of males with continence control concerns. Currently males have several options, from catheters and adult diapers, to pouches and pads.

Catheters are invasive, and painful to many recipients. Furthermore, outside a hospital setting, catheters decrease the mobility of a person and make traveling more cumbersome and difficult.

Thousands have turned to adult diapers for their continence concerns. For males with only minor incontinence, such as dripping urine, diapers are overkill. Some adults who need protection simply refuse to purchase diapers for their own personal reasons. Many of these people associate diapers only with children. Some find diapers bulky and/or noisy. Many feel the bulkiness identifies the wearer to the general public as one with continence control problems. Whether this perception by the wearer is justified or not is unimportant, for the emotional state of some users of adult diapers is affected. The process of changing adult diapers, whether by the wearer of caretaker, can be cumbersome, especially in public. Finally, because of the size and bulkiness of adult diapers, traveling with extra diapers is restrictive.

Pads or sanitary napkins also have several shortfalls. Protection is dependent on the position of the penis. Some associate pads only with women, and will find these embarrassing to use.

Pouches only covering the penis shaft have been invented. These are an improvement over the previous stated options. However, these pouches are a “one size fits all continence problems”, thus more bulky than many would like. Adjustable tabs for different penis sizes add to the manufacturing cost of the pouch, which is then passed on to the consumer. Finally, adjustable tabs only secure the pouch to the penis under the tabs, and a snug fit along the entire penis is not achieved.

Prior related inventions are the following:

Nilsson, U.S. Pat. No. 6,138,678, Male incontinence guard

Nygren, U.S. Pat. No. 6,105,174, Male incontinence pouch

Dann, U.S. Pat. No. 5,752,944, Male incontinence device

Ishikawa, U.S. Pat. No. 5,735,837, Urine-absorbent bag for incontinence

Duperret, U.S. Pat. No. 5,695,485, male continence pouch and shield.

Grizzaffi, U.S. Pat. No. 5,275,592, athletic supporter attached to pouch.

Bryant, U.S. Pat. No. 5,074,853, a penis diaper with elastic band.

Dahlgren, U.S. Pat. No. 4,901,375, a pouch holding a plastic urinal bottle.

Runeman, U.S. Pat. No 4,710,188, a bag which receives the penis and scrotum.

Garfinkel, U.S. Pat. No. 3,616,789, an incontinent garment.

### SUMMARY OF THE INVENTION

The Disposable Bladder permits a male to urinate in a watertight reservoir while sleeping, at home, or in public, without wetting himself. This invention lets the wearer choose the appropriate size for his level of incontinence, with the minimum of discomfort and obtrusiveness. This is a lightweight, inexpensive, and convenient alternative to other products on the market.

The urine enters the reservoir through a tube that surrounds the penis. The reservoir of the disposable bladder will vary in size. A small reservoir can be used when urine drips from the penis at a slow rate. A wearer can choose a mid-size reservoir for mild bladder control problems. A large reservoir can be used when little or no bladder control exists. Thus, a wearer can purchase the appropriate size “Disposable Bladder” for his needs. This is especially important for people with minor dripping problems. The overkill issue with “one size for all” pouches and diapers is addressed here. A person with a minor problem does not need to purchase a bulkier product made to handle all incontinence problems. Now a wearer can choose a device that is tailored to his incontinence level.

“The Disposable Bladder” attaches to the wearer in the same fashion a condom attaches to the wearer. An elastic tube that is connected to the reservoir rolls out over the penis to accommodate different penis lengths, thus assuring a snug fit along the entire penis. An elastic tube also ensures a snug fit on penis shafts of different circumferences. This eliminates the need for adjustable tabs to accommodate different penis sizes. The tube that surrounds the penis is not only elastic, but is permeable, allowing for the penis shaft to stay cool and dry of perspiration. Thus, the reservoir does not surround the penis; only the tube that connects to the reservoir surrounds the penis. This design is simple, and allows for a snug, comfortable fit along the entire penis.

The reservoir consists of three layers. As with many diapers on the market, the outer layer of the reservoir is impermeable. The middle layer contains a sponge like substance that absorbs the urine. The inner layer is permeable and non-absorbent and protects any contact of the penis with the urine absorbing middle layer, yet allows the urine to pass through it into the middle layer. When the middle layer is full of urine, the disposable bladder can be discarded in the toilet or trash. For the larger reservoirs, an adhesive material can be placed on the outside of the reservoir to adhere to one's clothes. This will keep the disposable bladder secure and in the groin area, and any discomfort from the weight of the urine-filled bladder will be diminished.

“The Disposable Bladder” can be manufactured in the same manner that diapers are manufactured, and can be made of the same or like materials as diapers. The best mode contemplated by this inventor to bring “The Disposable Bladder” to the public at large would be for this inventor to enter into a partnership with a major diaper manufacturer for the production and/or marketing of “The Disposable Bladder”.

### BRIEF DESCRIPTION OF THE DRAWING

FIG. 1 is an overhead view of the device, laid from the open end to the closed end.

### DETAILED DESCRIPTION OF THE INVENTION

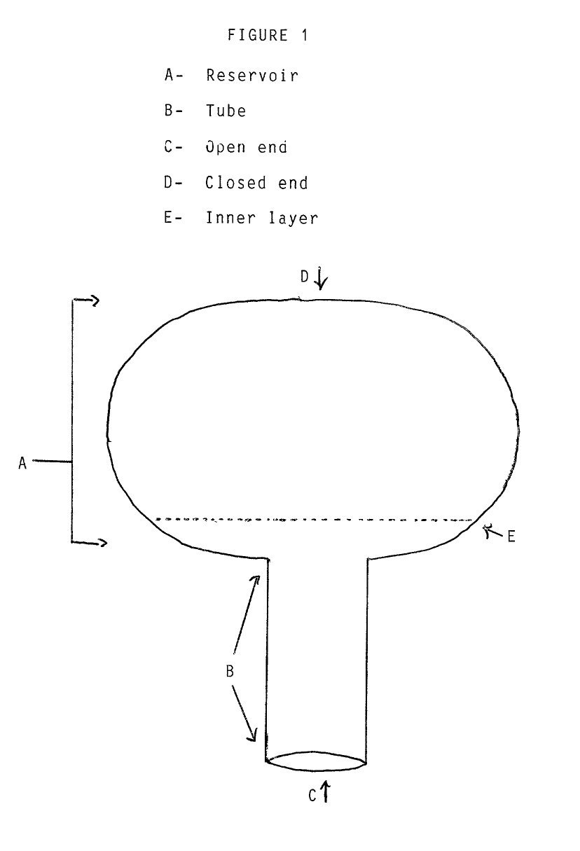

FIG. 1 is an overhead view of “The Disposable Bladder”. Reservoir (A) contains an outer layer, a middle layer, and an inner layer. The tube (B) shown is in its rolled out extended position. The penis enters the open end (C) of the rolled up elastic tube and then the tube is rolled out over the penis by the wearer in the same manner as a condom. The dotted line (E) depicts the non-absorbent, permeable inner layer, which can't be seen from the outside. This layer allows urine to enter the middle layer, while protecting the penis from contact with urine absorbed in the middle layer. The middle layer is the section between the closed end (D) and the inner layer (E), and is filled with highly absorbent materials. The middle layer is not depicted in FIG. 1. The impermeable outer layer lines the outside of the reservoir (A) and keeps the urine contained in the reservoir. Reservoir (A) can be purchased in different sizes, based on the wearer's level of incontinence control.

1. I claim a disposable device for male incontinence, consisting of
1) an open end;
2) a closed end;
3) an elastic permeable tube which receives the penis in the open end;
4) a reservoir at the closed end by which urine enters via the tube;
5) a reservoir with an outer impermeable layer;
6) a reservoir with a middle layer containing highly absorbent materials;
7) a reservoir with a permeable, non-absorbent inner layer;
8) adhesive strips on the outside surface of the reservoir;
